(19) United States
(12) Patent Application Publication
(10) Pub. No.: US 2019/0160126 A1
(43) Pub. Date: May 30, 2019

(54) COMPOSITIONS AND METHODS FOR TREATING REPRODUCTIVE INDICATORS SYMPTOMATIC OF MALE INFERTILITY

(71) Applicant: Omar Saleh ALAFFARI, Joplin, MO (US)

(72) Inventor: Omar Saleh ALAFFARI, Joplin, MO (US)

(21) Appl. No.: 15/824,219

(22) Filed: Nov. 28, 2017

(51) Int. Cl. A61K 36/31; A61K 36/48; A61K 36/328; A61K 36/71; A61K 9/16; A23L 2/52; A23L 33/105; A23L 33/00

(52) U.S. Cl. CPC A61K 36/31; A61K 36/48; A61K 36/328; A61K 36/71; A23V 2002/00; A23L 2/52; A23L 33/105; A23L 33/40; A23L 33/30; A61K 9/16

(57) ABSTRACT

A nutraceutical composition containing (i) garden cress, (ii) nigella saliva, (iii) commiphora myrrha, and (iv) fenugreek for treating male infertility and a method of treating symptomatic reproductive indicators of male infertility such as sperm concentration, sperm count, and progressive sperm motility by administering an effective amount of the nutraceutical composition having an effective proportion of each constituent.

### GRANT OF NON-EXCLUSIVE RIGHT

This application was prepared with financial support from the Saudi Arabian Cultural Mission, and in consideration therefore the present inventor(s) has granted The Kingdom of Saudi Arabia a non-exclusive right to practice the present invention.

### BACKGROUND OF THE INVENTION

### Technical Field

The present disclosure relates to a nutraceutical composition for treating male infertility and a method of using thereof for increasing at least one reproductive indicator selected from the group consisting of sperm concentration, sperm count, and progressive sperm motility.

### Description of the Related Art

The “background” description provided herein is for the purpose of generally presenting the context of the disclosure. Work of the presently named inventors, to the extent it is described in this background section, as welt as aspects of the description which may not otherwise qualify as prior art at the time of filing, are neither expressly or impliedly admitted as prior art against the present invention.

Infertility is a serious social problem facing developed countries today as the birth rate declines and the number of infertile couples increases. Approximately 15% of couples attempting their first pregnancy do not succeed. Most authorities define these individuals as infertile if they have been unable to achieve a pregnancy after one year of unprotected intercourse. Conception normally is achieved within twelve months in 80-85% of couples who use no contraceptive measures, and persons not conceiving after this time should therefore be regarded as possibly infertile and should seek evaluation. The available data over the past twenty years suggest that in approximately 30% of cases pathology is found in the man alone, and in another 20% both the man and woman show abnormalities. Therefore, the male factor is at least partly responsible in about 50% of infertile couples.

Sperm are produced by repeated division of cells in small coiled tubules with the testes at a rate of approximately 100 million per day. Sperm production is a lengthy process, from the the beginning of division of the stem cell to the appeance of mature sperm in the semen takes about 3 months. The sperm spend 2 to 10 days passing through the epididymis, during which time they mature and become capable of swimming and ferilizing eggs. The volume of liquid from the testes and epididymides is less than 5% of the total semen volume. About 65% of the semen volume comes from the seminal vesicles and 25% from the prostate gland. The average semen volume for healthy men ejaculating every two days is 3 ml with a sperm concentration of approximately 85 million per mL. During ejaculation the sperm and the prostatic fluid come out first and the seminal vesicle fluid follows. The seminal vesicle fluid cagulates giving the sememn a lumpy gel-like appearance. After approximately 10 minutes or so liquefication occurs and the gel disappears. Male infertility is commonly due to deficiencies in the semen, and semen quality may be used as a surrogate measure of male fecundity.

Male infertility may be caused by many conditions that affect the production of functional sperm. The most common cause is varicocele (i.e. a hardening of the veins that drain the testes) which accounts for about 40% of cases and may be treated surgically. Testicular failure accounts for approximately 10% of cases and may result from numerous causes including, but not limited to, malignancy, mumps, Kleinfelter's syndrome, injuy, and radio- or chemotherapy. Hyperspermia, increased seminal fluid volume, also accounts for about 10% of cases. Endocrine disease affecting spermatogenesis account for approximately 9% of cases and typically involve pituitary or adrenal hypoplasia or hyperthyroidism. Ovstruction of the ejaculatory duct accounts for about 5% of cases and sperm autoantibodies for 1 to 2%.

Approximately 13% of afflicted men have untreatable sterility, 11% have treatable conditions and 76% have disorders of sperm production or function which do not usually have clearly defined effective treatments. Of the 13% of afflicted men with untreatable sterility, most have no sperm in their semen (azoospermia) because the tubules in the testes which produce sperm did not develop or have been irreversibly damaged. The may be associated with filure of the testes to descend into the scrotum during childhood, inflammation of the testes and or treatment with certain drugs. In some sterile men, sperm are produced in normal numbers, but they are either not motile (i.e. do not swim) or lack structures necessary for penetration and fertilization of eggs which may be detected by microscopic examination of the shape of the sperm and reported as abnormal morphology. Some men with failure of sperm production do not produce nomal amounts of the male sex hormone, testosterone, and their general health and sexual performance is improved by treatment with testosterone. Over three quarters of men investigated for infertility have sperm in the semen, but in lower numbers than normal (i.e. oligospermia, ˜38%), or in adequate numbers but with reduced motility (˜33%).

Oligospermia is a condition associated with an abnormally low number of sperm in the ejaculate of the male. The normal range of sperm count is between 20 million/mL and 200 million/mL. The finding that the sperm count is below 20 million/mL indicates oligospermia. Common cause of oligospermia include, but are not limited to, stress, smoking of tobacco (nicotine damages sperm), lead (workers in printing presses have lowered spend counts), hot climates, saunas, hot baths, the wearing of tight underwear, and other conditions and situation in which scrotal temperature may be raised, varicocele an increased consumption of alcohol. In addition, certain medications, for example, antipsychotic drugs used for treating schizophrenia such as risperidone, often result in sexual dysfunction and/or reduced fertility in male animals (i.e. dogs, cats, horses, mice, rate) and male patients.

Accordingly, there exists a considerable need for safe agents that can effectively treat and/or manage the many symptoms and causes of male infertility. Nutraceuticals, in recent decades, have emerged as an increasingly popular option for the treatment and prevention or various diseases. Nutraceuticals, a term coined as a portmanteau of the words “nutrition” and “pharmaceutical”, refers to products that range from isolated nutrients todietary supplements and herbal products that provide medical and health benefits including prevention and treatment of disease [Kalra, E K, 2003, “Nutraceutical—definition and introduction,” AAPS pharmSci 5(3): 27-28; Ramaa, C S, Shirode A R, Mundada A S, and Kadam V J, 2006, “Nutraceuticals—an emerging era in the treatment and prevention of cardiovascular diseases,” Curr Pharm Biotechnol 7(1): 15-23—each incorporated herein by reference in its entirety].

In view of the forgoing, one object of the present disclosure is to provide nutraceutical compositions and beverages thereof comprising (i) garden cress, (ii) nigella saliva, (iii) commiphora myrrha, and (iv) fenugreek suitable for treating male infertility. A further aim of the present disclosure is to provide methods for treating symptomatic reproductive indicators of male infertility, such as sperm concentration, sperm count, and progressive sperm motility by administering an effective amount of the nutraceutical composition having an effective proportion of each constituent.

### BRIEF SUMMARY OF THE INVENTION

According to a first aspect, the present disclosure relates to a nutraceutical composition for treating male infertility, the composition comprising (i) garden cress, (ii) nigella sativa, (iii) commiphora myrrha, and (iv) fenugreek, wherein each constituent (i), (ii), (iii), and (iv) is present in an effective amount and an effective proportion relative to the total amount of (i)-(iv) such that when administered to a male human subject in need thereof the nutraceutical composition is effective in increasing at least one reproductive indicator selected from the group consisting of sperm concentration, sperm count, and progressive sperm motility relative to the reproductive indicator prior to the administering.

In one embodiment, the nutraceutical composition further comprises (v) a pharmaceutically acceptable carrier or excipient.

In one embodiment, the nutraceutical composition has a wet basis moisture content of less than 15% relative to the total weight of the nutraceutical composition.

In one embodiment, the nutraceutical composition is in a powder form and has an average particle size of 0.1-2.5 mm.

In one embodiment, a weight ratio of garden cress to nigella sativa to commiphora myrrha to fenugreek is in the range of 5-10:5-10:0.25-2.0:2-6.

In one embodiment, a weight ratio of garden cress to nigella sativa to commiphora myrrha to fenugreek is 7:7:1:4.5.

According to a second aspect, the present disclosure relates to a method of increasing at least one reproductive indicator selected from the group consisting of sperm concentration, sperm count, and progressive sperm motility, the method comprising administering to a male human subject in thereof an effective dosage of a nutraceutical composition to increase at least one reproductive indicator selected from the group consisting of sperm concentration, sperm count, and progressive sperm motility, the nutraceutical composition comprising (i) garden cress, (ii) nigella sativa, (iii) commiphora myrrha, and (iv) fenugreek, wherein each constituent (i), (ii), (iii), and (iv) is present in an effective amount and an effective proportion relative to the total amount of (i)-(iv) such that when administered to the male human subject the nutraceutical composition is effective in increasing at least one reproductive indicator selected from the group consisting of sperm concentration, sperm count, and progressive sperm motility relative to the reproductive indicator prior to the administering.

In one embodiment, the nutraceutical composition further comprises (v) a pharmaceutically acceptable carrier or excipient.

In one embodiment, the nutraceutical composition has a weight ratio of garden cress to nigella sativa to commiphora myrrha to fenugreek in the range of 5-10:5-10:0-25-2.0:2-6.

In one embodiment, the nutraceutical composition has a weight ratio of garden cress to nigella sativa to commiphora myrrha to fenugreek of 7:7:1:4.5.

In one embodiment, the administering is performed in an enteral manner.

In one embodiment, the nutraceutical composition is administered in the form of an encapsulated powder.

In one embodiment, the nutraceutical composition is administered at a dosage in the range of 1-10 mg at least once per day.

In one embodiment, the administering is performed 1-5 times daily.

In one embodiment, the administering is performed 1-5 times daily for a time period of 5-150 days.

In one embodiment, the method increases at least one reproductive indicator in the mate human subject selected from the group consisting of sperm concentration, sperm count, and progressive sperm motility by 40-80% relative to the at least one reproductive indicator in the male human subject selected from the group consisting of sperm concentration, sperm count, and progressive sperm motility prior to the administering.

According to a third aspect, the present disclosure relates to a nutraceutical beverage for treating male infertility, the beverage comprising (i) garden cress, (ii) nigella sativa, (iii) commiphora myrrha, (iv) fenugreek, and (vi) a liquid, wherein each constituent (i), (ii), (iii), and (iv) is present in an effective amount and an effective proportion relative to the total amount of (i)-(iv) such that when administered to a male human subject in need thereof the nutraceutical beverage is effective in increasing at least one reproductive indicator selected from the group consisting of sperm concentration, sperm count, and progressive sperm motility relative to the reproductive indicator prior to the administering.

In one embodiment, a weight ratio of garden cress to nigella sativa to commiphora myrrha to fenugreek is in the range of 5-10:5-10:0.25-2.0:2-6.

In one embodiment, a weight ratio of garden cress to nigella sativa to commiphora myrrha to fenugreek is 7:7:1:4.5.

In one embodiment, a ratio of total weight of (i)-(iv) in grams to total volume of the nutraceutical beverage is in the range of 1:5 to 1:100.

The foregoing paragraphs have been provided by way of general introduction, and are not intended to limit the scope of the following claims. The described embodiments, together with further advantages, will be best understood by reference to the following detailed description.

### DETAILED DESCRIPTION OF THE EMBODIMENTS

Embodiments of the present disclosure will now be described more fully hereinafter, in which some, but not all of the embodiments of the present disclosure are shown.

The present disclosure will be better understood with reference to the following definitions.

As used herein, the words “a” and “an” and the like carry the meaning of “one or more”. Within the description of this disclosure, where a numerical range is stated, the endpoints are included unless stated otherwise. Also, all values and subranges within a numerical limit or range are specifically included as if explicitly written out.

According to a first aspect the present disclosure relates to a nutraceutical composition for treating male infertility, the composition comprising (i) garden cress, (ii) nigella sativa, (iii) commiphora myrrha, and (iv) fenugreek, wherein each constituent (i), (ii), (iii), and (iv) is present in an effective amount and an effective proportion relative to the total amount of (i)-(iv) such that when administered to a male human subject in need thereof the nutraceutical composition is effective in increasing at least one reproductive indicator selected from the group consisting of sperm concentration, sperm count, and progressive sperm motility relative to the reproductive indicator prior to the administering.

As used herein, the terms “treat”, “treatment” and “treating” in the context of the administration of a therapy to a subject in need thereof refer to the reduction or inhibition of the progression and/or duration of a disease or condition (e.g. male infertility), the reduction or amelioration of the severity of the disease or condition, and/or the amelioration of one or more symptoms thereof (e.g. increasing a reproductive indicator such as for example perm concentration, sperm count, and progressive sperm motility) resulting from the administration of one or more therapies or compositions. As used herein, “treating” or “treatment” of the disease or condition includes preventing the disease or condition from occurring in a subject that may be predisposed to the disease or condition but does not yet experience or exhibit symptoms associated with the disease or condition (prophylactic treatment), inhibiting the disease or condition (slowing and/or arresting its development), ameliorating the disease or condition, providing relief from the symptoms or side-effects of the disease or condition (including palliative treatment), and relieving the disease or condition (causing regression of the disease or condition).

With regard to the disease or condition, these terms simply mean that one or more of the symptoms of the disease or condition, such as male infertility, will be reduced. As used herein, male infertility refers to a male's inability to cause pregnancy in a fertile female. Male infertility is commonly due to deficiencies in the semen, and semen quality may be used as a surrogate measure of male fecundity. Factors that relate to male infertility include pre-testicular causes, testicular factors, and post-testicular cause. Pre-testicular causes refer to conditions that impede adequate support of the testes and include incidents of poor hormonal support and poor general health including, but not limited to, hyporgonadotropic hypogonadism, obesity, undiagnosed and/or untreated coeliac disease (CD), drugs, alcohol, strenuous riding, medications, including, but not limited to, those that affect spermatogenesis such as chemotherapy, anabolic steroids, cimetidine, spironolactone, those that decrease follicle-stimulating hormone (FSH) such as phenytoin, those that decrease sperm motility such as sulfasalazine and nitrofurantoin, genetic abnormalities such as Robertsonian translocation, tobacco smoking, DNA damage and epigenetic conditions. Testicular factors refer to conditions where the testes produce semen of low quantity and/or poor quality despite adequate hormonal support and include, but are not limited to, varicocele, age, genetic defects on the Y chromosome, chromosomal abnormalities (i.e. Klinefelter syndrome), neoplasm (i.e. seminoma), idiopathic failure, cryptorchidism, trauma, hydrocele, mumps, malaria, testicular cancer, defects in peptidase enzyme USP26, acrosomal defects affecting egg penetration, and radiation therapy. Post-testicular causes decrease male fertility due to conditions that affect the male genital system after testicular sperm production and include defects of the genital tract as well as problems with ejaculation. Exemplary post-testicular causes include, but are not limited to, Vas deferens obstruction, lack of Vas deferens, infection (i.e. prostatitis), retrograde ejaculation, ejaculatory duct obstruction, hypospadias, and impotence. In terms of the present disclosure, male infertility may refer to the disease or condition caused by pre-testicular causes, testicular factors, post-testicular causes, and mixtures thereof, preferably pre-testicular causes and testicular factors. In preferred embodiments, male infertility refers to lower numbers of sperm in the semen (oligospermia) and/or adequate numbers of sperm in the semen but with reduced motility.

As used herein, a subject in need thereof or in need of treatment includes, but is not limited to, a subject already with the disease or condition, a subject which does not yet experience or exhibit symptoms of the disease or condition, and a subject predisposed to the disease or condition. As used herein, the terms “patient”, “subject”, and “individual” are used interchangeably. As used herein, these terms refer to individuals suffering from a disease or condition, and encompass mammals and non-mammals, preferably mammals, preferably humans, preferably male humans. None of the terms require the individual be under the care and/or supervision of a medical professional. As used herein, mammals are any members of the mammalian class and are not limited to humans, exemplary mammals include, but are not limited to, non-human primates, such as chimpanzees, and other apes and monkey species, farm animals, such as cattle, horses, sheep, goats, swine, domestic animals, such as rabbits, cats, and dogs, laboratory animals including rodents, such as rates, mice and guinea pigs, and the like. Examples of non-mammals include, but are not limited to, birds, fishes, and the like. In preferred embodiments of the compositions and methods described herein, the subject is a mammal, preferably the subject is a human, and most preferably the subject is a male human. In certain embodiments, a subset of the human population may be predisposed to male infertility.

As used herein, the terms “therapies” and “therapy” can refer to any method, composition, and/or active ingredient that can be used in the treatment and/or management of the disease or condition and/or one or more symptoms thereof. In certain embodiments, the method for treating the disease or condition involves the administration of a unit dosage or a therapeutically effective amount of a composition thereof to a subject in need thereof. As used herein, the term “active ingredient” refers to an ingredient or constituent in the nutraceutical composition as described herein in any of its embodiments that is biologically active, such as, for example the nutraceutical composition itself described herein, in any of its embodiments.

As used herein, the terms “effective amount”, “effective proportion”, “therapeutically effective amount”, “therapeutically effective proportion” or “pharmaceutically effective amount” and “pharmaceutically effective proportion” refer to that amount of the nutraceutical composition described herein in any of its embodiments being administered which will relieve to some extent one or more of the symptoms of the disease or condition being treated. The result can be reduction and/or alleviation of the signs, symptoms, or causes of a disease, or any other desired alteration of a biological, such as, for example an increase in a reproductive indicator. For example, an effective amount or effective proportion for therapeutic uses may be the amount of the nutraceutical composition and/or the amount of its constituents (i)-(iv) or derivatives and combinations thereof as disclosed herein required to provide a clinically significant decrease in the disease or condition. An appropriate effective amount or effective proportion may differ from one individual to another. In certain embodiments, an appropriate effective amount or effective proportion in any individual case may be determined using techniques, such as, for example, a dose escalation study.

In a preferred embodiment, each constituent garden cress (i), nigella sativa (ii), commiphora myrrha (iii), and fenugreek (iv) is present in an effective amount and an effective proportion relative to the total amount of (i)-(iv) such that when administered to a male human subject in need thereof the nutraceutical composition is effective in increasing at least one reproductive indicator selected from the group consisting sperm concentration, sperm count, and progressive sperm motility. In certain embodiments, the nutraceutical composition enhances the fertility of a male individual by increasing the number of spermatozoa, and/or enhancing the motility of spermatozoa, and/or adjusting the volume of the ejaculate and or adjusting the liquefaction time of the ejaculate. It is equally envisaged that the nutraceutical composition may be effective in improving other symptomatic reproductive indicators associated with male infertility in addition to or in lieu of sperm concentration, sperm count, and or sperm motility including, but not limited to, ejaculate volume, abnormal ejaculate appearance, abnormal ejaculate viscosity, abnormal ejaculate color, abnormal ejaculate pH, ejaculate liquefaction time, ejaculate percent motility, quality of the motility (i.e. decreased none progressive motility, decreased immotility), quality of the morphology (i.e. increased normal forms, decreased abnormal forms such as abnormal heads, midpieces, and principle pieces). Suitable methods for evaluating reproductive indicators of male infertility are well known to those of ordinary skill in the art. It is equally envisaged that the nutraceutical composition of the present disclosure in any of its embodiments may have additional health benefits in addition to male infertility including, but not limited to, appetite suppression, effecting the intestinal flora, promotion of lean body mass, antidepressant effects, reduced body fat, and the like.

As used herein, a “composition” refers to an active ingredient itself, a mixture of multiple active ingredients, and mixtures with other chemical or natural components designed to facilitate administration of the nutraceutical composition and active ingredient(s) thereof to a subject. As used herein, a “nutraceutical” is a portmanteau of the words “nutrition” and “pharmaceutical” and the term refers to and is applied to products that range from isolated nutrients, dietary supplements and herbal products, specific diets, and processed foods. As used herein “nutraceutical” refers to a natural or synthesized substance that provides at least a perceived health or medical benefit, preferably natural. Nutraceuticals are products derived from food sources that are purported to provide extra health benefits, in addition to the basic nutritional value found in foods and may prevent chronic diseases, improve health, delay the ageing process, increase life expectancy, or support the structure or function of the body.

In certain embodiments, the nutraceutical composition may be considered a dietary supplement composition. As used herein, a “dietary supplement” refers to a product that contains nutrients derived from food products that are concentrated in liquid or capsule form, and further is a product preferably ingested, that contains one or more dietary ingredients intended to supplement the diet. These dietary ingredients may include, but are not limited to vitamins, minerals, herbs or other botanicals used as medicinal plants, amino acids, and substances such as enzymes, organ tissues, glandulars, and metabolites. Dietary supplements can also be extracts or concentrates and may be found in many forms such as tablets, capsules, softgels, gelcaps, liquids, and/or powders.

Herbal medicine, or herbalism, refers to the use of plants for medicinal purposes. As used herein, “plant” or “plant part” refers to any living organism of the kingdom Plantae and includes all plants described as grains, fruits and vegetables as well all plant parts or component including, but not limited to, roots, barks, branches, seeds, extracts, stems, stem shoots, leaves, bulbs, nuts, beans, grains, flowers, flower bud, pollen, vegetable oils, vegetable skins, vegetable fats, fruits and fruit skins during all periods of growth, preferably roots, barks, branches, seeds, extracts, stems, and leaves during all periods of growth. In terms of the present disclosure, the plant is for example selected from the group including, but not limited to, herbs, medicinal plants, teas, and spices. In general, “herbs” refer to the leafy and/or green parts of a plant (either fresh or dried) in contrast to a “spice” which refers to a product from another part of the plant (usually dried) including, but not limited to, seeds, berries, bark, roots and fruits. In terms of the present disclosure, garden cress (i), nigella sativa (ii), commiphora myrrha (iii), and fenugreek (iv) refer to the plant and/or all constituent plant parts during all periods of growth.

In one embodiment, the nutraceutical composition comprises garden cress (i) plant and/or plant parts. Garden cress (Lepidium sativum or cress) is a fast growing edible herb botanically related to watercress and mustard and sharing their peppery, tangy flavor and aroma. It is considered an important medical plant and the whole plant, leaves, root and seed can be used for a multitude of medicinal purposes. For example, the seed and its oil, in particular have been found useful in the treatment of asthma, coughs, bronchitis and the improvement of lung function. In certain regions, garden cress may be known as or referred to as cress, mustard and cress, garden pepper cress, pepperwort or pepper grass. Garden cress is a green perennial plant used as a leaf vegetable consumed by humans typically as a garnish This annual plant has an average height of 20-100 cm with many branches on the upper part and when mature produces white to pinkish flowers that are 0.5-5.0 mm across clustered in branched racemes and small seedpods. When consumed raw cress is a high-nutrient food containing substantial content of vitamins including, but not limited to, A, C, and K and several dietary minerals including, but not limited to, manganese, calcium iron, magnesium, phosphorous, and potassium. Garden cress oil is obtained from garden cress seeds by methods including, but not limited to, cold pressing, hydraulic pressing, solvent extraction, and supercritical CO₂.

In one embodiment, the nutraceutical composition comprises nigella sativa (ii) plant and/or plant parts. Nigella sativa (black-caraway, black cumin, fennel-flower, nutmeg flower, Roman-coriander, black seeds, nigella, Bunium bulbocastanum, and/or kulonji) is an annual flowering plant in the family Ranunculaceae native to south and southwest Asia. Nigella sativa grows to 20-30 cm tall, with finely divided, linear (but not thread-like) leaves. The flowers are delicate, and usually colored pale blue and white, with five to ten petals. The black caraway fruit is a large and inflated capsule composed of three to seven united follicles, each containing numerous seeds. Nigella sativa has a history of use in medicine and its efficacy has been investigated for use in asthma (both prevention and treatment of acute attacks), allergic rhinitis, atopic dermatitis, functional dyspepsia, respiratory problems, seizure disorders, diabetes mellitus and the metabolic syndrome, and opioid addiction.

In certain embodiments, nigella sativa may refer to the deep black, sharp-cornered seed grains of nigella sativa. Nigella sativa oil commonly contains conjugated limoleic (18:2) acid, thymoquinone, nigellone (dithymoquinone), melanthin, nigilline, and trans-anethole. Nigella sativa, the seed and/or its extracts may contain numerous esters of structurally unusual unsaturated fatty acids with terpene alcohols. In addition, the seeds may contain alkaloids which may belong to two different types, such as, for example isochinoline alkaloids represented by for example, nigellimin and nigellimin-N-oxide, and pyrazol alkaloids including, but are not limited to nigellidin and nigellicin. The seeds and the essential oil of the seeds may contain thymoquinone, p-cymene, pinene, dithymoquinone, thymohydroquinone, and mixtures thereof. In addition, the seeds and the essential oil may contain other terpene derivatives in low amounts including, but not limited to Carvacrol, carvone, limonene, 4-terpineol, and citronellol. Moreover, the seed and essential oil may contain amounts of fatty acid ethyl esters. In certain embodiments, storage of thmoquinone may yield dithymoquinonene and higher oligocondensation products (i.e. nigllone). The seeds may also contain a fatty oil rich in unsaturated fatty acids including, but not limited to, linoleic acid, oleic acid, eicodadienoic acid and dihomlinoleic acid characteristic for the genus. Further, the seeds may contain saturated fatty acids including, but not limited to palmitic acid and stearic acid.

In one embodiment, the nutraceutical composition comprises commiphora myrrha (iii), plant and/or plant parts. Myrrh is a natural gum or resin extracted from a number of small, thorny tree species of the genus Commiphorai and/or the family Burseraceae. Myrrh resin has been used throughout history as a perfume, incense, and medicine. Myrrh gum is commonly harvested from the species Commiphora myrrha. Another commonly used name, Commiphora molmol is a synonym as are commiphora myrrha, myrrh, African myrrh, herabol myrrh, Somali myrrhor, mommon myrrh, or gum myrrh. Commiphora myrrha is native to Yemen, Somalia, Eritrea and eastern Ethiopia. As used herein, the definition also includes several oleo gum resins of a number of other Commiphora species and myrrh-like resins including, but not limited to, opopanax, balsam, bdellium, guggul bisabol, and Indian myrrh. In pharmacy, myrrh is used as an antiseptic in mouthwashes, gargles, and toothpastes. Myrrh is currently used in some liniments and healing salves that may be applied to abrasions and skin ailments and it has also been recommended as an analgesic. Additionally, myrrh gum is used for indigestion, ulcers, colds, cough, asthma, lung congestion, arthritis pain, and some cancers.

Commiphora myrrha may contain volatile oils (i.e. heerabolene, cadiened, elemol, eugenol, cuminaldehyde, sesquiterpenes such as furanoeudesma-1,3-diene and curzarene, and numerous furanosequiterpenes such as furanodiene, furanodienone, curzerenone, lindestrene, 2-methoxyfuranodiene and other derivatives), resins (i.e. alpha, beta, and gamma commiphoric acids, commiphorinic acid, herraboresene, alpha and beta heerabomyrrhols, and commiferin), and gums (i.e. composed of arabinose, galactose, xylose, and 4-0-methylglucuronic acid.

In one embodiment, the nutraceutical composition comprises fenugreek (iv) plant and/or plant parts. Fenugreek (Trigonella foenum-graecum) is an annual plant in the family Fabaceae, with leaves consisting of three small obovate to oblong leaflets. It is cultivated worldwide as a semiarid crop, and its cuboid-shaped and yellow to amber colored seeds are a common ingredient in dishes popular in the Indian subcontinent. Often, fenugreek is used as an herb (dried or fresh leaves), spice (seeds), and vegetable (fresh leaves, sprouts, and microgreens). Its tender leaves are used as vegetables and its seeds as condiments. Sotolon is the chemical largely responsible for the distinctive sweet smell of fenugreek. Debitterisation of the fenugreek seed does not compromise its hypocholesterolemic and hypoglycemic properties. Fenugreek seeds are rich sources of proteins, dietary fiber, B vitamins, iron and sever other dietary minerals including, but not limited to, calcium and phosphorous. The seeds of fenugreek additionally contain fiber including mucilage, hemicellulose, cellulose, and lignin, as well as, steroid saponins, lipids and trigoneline.

In a preferred embodiment, the nutraceutical composition of the present disclosure in any of its embodiments has a weight ratio of garden cress to commiphora myrrha to fenugreek in the range of 5-10:5-10:0.25-2.0:2-6, preferably 6-9:5-10:0.25-2.0:2-6, preferably 6.5-8.5:5-10:0.25-2.0:2-6, preferably 7:5-10:0.25-2.0:2-6, preferably 5-10:6-9:0.25-2.0:2-6, preferably 5-10:6.5-8.5:0.25-2.0:2-6, preferably 5-10:7:0.25-2.0:2-6, preferably 5-10:5-10:0.5-1.5.0:2-6, preferably 5-10:5-10:0.75-1.25:2-6, preferably 5-10:5-10:1:2-6, preferably 5-10:5-10:0.25-2.0:3-5.5, preferably 5-10:5-10:0.25-2.0:4-5, preferably 5-10:5-10:0.25-2.0:4.5. In a preferred embodiment, the nutraceutical composition of the present disclosure in any of its embodiments has a weight ratio of garden cress to commiphora myrrha to fenugreek in the range of 5-10:5-10:0.25-2.0:2-6, preferably 6-9:6-9:0.5-1.5:3-5.5, preferably 6.5-8.5:6.5-8.5:0.75-1.25:4-5, most preferably 7:7:1:4.5.

In a preferred embodiment, the nutraceutical composition of the present disclosure in any of its embodiments comprises 20-60% by weight of garden cress relative to the total weight of the nutraceutical composition, preferably 25-55 wt %, preferably 30-50 wt %, preferably 32-45 wt %, preferably 34-40 wt %, or about 36 wt % of garden cress relative to the total weight of the nutraceutical composition. In a preferred embodiment, the nutraceutical composition of the present disclosure in any of its embodiments comprises 20-60% by weight of nigella sativa relative to the total weight of the nutraceutical composition, preferably 25-55 wt %, preferably 30-50 wt %, preferably 32-45 wt %, preferably 34-40 wt %, preferably 35-38% or about 36 wt % of nigella sativa relative to the total weight of the nutraceutical composition. In a preferred embodiment, the nutraceutical composition of the present disclosure in any of its embodiments comprises 0.1-15% by weight of commiphora myrrha relative to the total weight of the nutraceutical composition, preferably 0.5-12 wt %, preferably 1-10 wt %, preferably 28 wt %, preferably 4-6 wt %, or about 5 wt % of commiphora myrrha relative to the total weight of the nutraceutical composition. In a preferred embodiment, the nutraceutical composition of the present disclosure in any of its embodiments comprises 5-40% by weight of fenugreek relative to the total weight of the nutraceutical composition, preferably 10-35 wt %, preferably 12-30 wt %, preferably 15-28 wt %, preferably 18-25 wt %, or about 23 wt % of fenugreek relative to the total weight of the nutraceutical composition. Most preferably, the nutraceutical composition comprises 30-40 wt % garden cress, 30-40 wt % nigella sativa, 1-10 wt % commiphora myrrha, and 20-25 wt % fenugreek, all relative to the total weight of the nutraceutical composition.

In certain embodiments, the nutraceutical composition of the present disclosure in any of its embodiments and/or its components may be dried. As used herein, wet basis moisture content refers to the percentage equivalent of the ratio of the weight of water to the total weight of the material. Often, wet basis moisture is used to describe the water content of agricultural materials and food products. In a preferred embodiment, the nutraceutical composition of the present disclosure in any of its embodiments and/or its components has a wet basis moisture content of less than 15% relative to the total weight of the nutraceutical composition and/or the component, preferably less than 12%, preferably less than 10%, preferably less than 8%, preferably less than 6%, preferably less than 4%, preferably less than 3%, preferably less than 2%, preferably less than 1% relative to the total weight of the nutraceutical composition. Drying may be advantageous to ensure the precision in the preparation of the dosage of the composition described herein and/or aid uptake by a subject's body. Means of drying are not viewed as particularly limiting and are well known to those of ordinary skill in the art. Exemplary means of drying include, but are not limited to direct solar drying, draining on absorbent towels, non-solar air drying, microwave oven drying, conventional oven drying, or drying agents such as silica gel or non-iodized table salt. Preferably, the nutraceutical composition and/or its components are dried in air at room temperature (i.e., about 22° C.).

In certain embodiments, the nutraceutical composition of the present disclosure in any of its embodiments and/or its components may be in a powder form, more preferably in a ground powder form. In a preferred embodiment, the nutraceutical composition of the present disclosure in any of its embodiments and/or its components may be in a powder form which has an average particle size of 0.1-2.5 mm, preferably 0.2-2.25 mm, preferably 0.3-2.0 mm, preferably 0.4-1.75 mm, preferably 0.5-1.5 mm, preferably 0.6-1.25 mm, preferably 0.8-1.0 mm, for example 0.2-1.0 mm, or about 0.38-1.0 mm. In certain embodiments, the nutraceutical composition of the present disclosure in any of its embodiments and/or its components is in a powder form which has a particle size distribution ranging from 33% of the average particle size to 133% of the average particle size, preferably 50-130%, preferably 60-125%, preferably 80-100%, preferably 90-110%, preferably 95-105% of the average particle size. In certain embodiments, the nutraceutical composition of the present disclosure in any of its embodiments and/or its components is in a powder form which are monodisperse, having a coefficient of variation or relative standard deviation, expressed as a percentage and defined as the ratio of the particle size standard deviation (σ) to the particle mean size (μ) multiplied by 100 of less than 25%, preferably less than 20%, preferably less than 15%, preferably less than 12%, preferably less than 10%, preferably less than 8%, preferably less than 6%, preferably less than 5%, preferably less than 2%. It is advantageous to control particle characteristics and ensure the success of nutraceutical powder processing and aid absorption once ingested. Means of grinding are not viewed as particularly limiting and are well known to those of ordinary skill in the art. Exemplary means of grinding include, but are not limited to mechanical grinding (i.e. mills, ball mills, rod mills, autogenous mills, SAG mills, pebble mills, high pressure grinding mills, buhrstone mills, vertical shaft impactor mills (VSI) tower mills) and hand grinding (i.e. a mortar and pestle).

In certain embodiments, the nutraceutical composition of the present disclosure in any of its embodiments may further comprise one or more medicinal plants in addition to components (i)-(iv). Exemplary medicinal plants include, but are not limited to. Açai (Euterpe oleracea), Alfalfa (Medicago sativa), Amica (Arnica Montana), Asthma weed (Euphorbia hirta). Astragalus (Astragalus propinquus), Barberry (Berberis vulgaris), Belladonna (Atropa belladonna), Bilberry (Vaccinium myrtillis), Bitter gourd (Momordica charantia), Bitter leaf (Vernonia amygdalina), Bitter orange (Citrus x aurantium), Black cohosh (Actaea racemosa), Blessed thistle (Cnicus benedicius), Blueberries (genus Vaccinium), Burdock (Arctium lappa), Cat's claw (Uncaria tomentosa), Cayenne (Capsicum annuum), Celery (Apium graveolens), Chamomile (Matricaria recutita and Anthemis nobilis), Chaparral (Larrea tridentata), Chasteberry (Vitex agnus-castus), Chili (Capsicum frutescens), Cinchona, Clove (Syzygium aromaticum), Coffee senna (Cassia occidentalis), Comfrey (Symphytum officinale), Cranberry (Vaccinium macrocarpon), Dandelion (Taraxacum officinale), Dong quai (Angelica sinensis), Elderberry (Sambucus nigra), Eucalyptus (Eucalyptus globulus), European Mistletoe (Viscum album), Evening primrose (Oenothera spp.). Feverfew (Tanacetum parthenium), Flaxseed (Linum usitatissimum), Garlic (Allium sativum), Ginger (Zingiber officinale), Gingko (Gingko biloba), Ginseng (Panax ginseng and Panax quinquefolius), Goldenseal (Hydrastis canadensis), Grape (Vitis vinifera), Guava (Psidium guajava), Hawthorn (specifically Crataegus monogyna and Crataegus laevigata), Hoodia (Hoodia gordonit), Horse chestnut (Aesculus hippocastanum), Horsetail (Equisetum arvense), Jamaica dogwood (Piscidia erythrina or Piscidia piscipula), Kava (Piper methysticum), Kha, Konjac (Amorphophallus konjac), Kratom (Mitragyna speciosa), Kanna (Sceletium tortuosum), Lavender (Lavandula angustifolia), Lemon (Citrus limon), Licorice root (Glycyrrhiza glabra), Marigold (Calendula officinalis), Marsh mallow (Althaea officinalis), Milk thistle (Stlybum marianum), Neem (Azadirachta indica), Noni (Morinda citrifolia), Oregano (Origanum vulgare), Papaya (Carica papaya), Peppermint (Mentha x piperita), Purple coneflower (Echinacea purpurea), Passion Flower (Passiflora), Red clover (Trifolium pratense), Rosemary (Rosmarinus officinalis), Sage (Salvia officinalis), Syrian Rue (aka Harmal) (Peganum harmala), St John's wort (Hypericum perforatum), Saw palmetto (Serenoa repens), Thunder God Vine (Tripterygium wilfordii), Thyme (Thymus vulgaris), Tulasi (Ocimum tenuiflorum or Holy Basil), Turmeric (Curcuma longa), Umckaloabo (Pelargonium sidoides), Valerian (Valeriana officinalis), White willow (Salix alba), Yerba santa (Eriodictyon crassifolium), and the like, and mixtures thereof.

In certain embodiments, the nutraceutical composition of the present disclosure in any of its embodiments may further comprise one or more culinary herbs and spices in addition to components (i)-(iv). Exemplary herbs and spices include, but are not limited to, Ajwain, carom seeds (Trachyspermum ammi), Akudjura (Solamum centrale), Alexanders (Smyrnium olusatrum), Alkanet (Alkanna tinctoria), Alligator pepper, mbongo spice (mbongochobi), hepper pepper (Aframomum danielli, A. citratum, A. exscapum), Allspice (Pimenta dioica), Angelica (Angelica archangelica), Anise (Pimpinella anisum), Aniseed myrtle (Syzygium anisatum), Annatto (Bixa orellana), Apple mint (Mentha suaveolens), Asafoetida (Ferula assafoerida), Asarabacca (Asarum europaeum), Avens (Geum urbanum), Avocado leaf (Peresea americana), Barberry (Berberis vulgaris and other Berberis spp), Basil, sweet (Ocimum basilicum), Basil, lemon (Ocimum x citriodorum), Basil, Thai (O.basilicum var. thyrsiflora), Basil, Holy (Ocimum tenuiflorum), Bay leaf (Laurus nobilis), Bay leaf, Indian, tejpat, malabathrum, Boldo (Peumus boldus), Borage (Baraga officinalis), Black cardamom (Amomum subulatum, Amomum costatum), Black mustard (Brassica nigra), Blue fenugreek, blue melilot (Trigonella caerulea), Brown mustard (Brassica juncea), Caraway (Carum carvi), Cardamom (Eleitaria cardamomum), Carob (Ceratonia siliqua), Catnip (Nepeta cataria), Cassia (Cinnamomum aromaticum), Cayenne pepper (Capsicum anmium), Celery leaf (Apium graveolens), Celery seed (Apium graveolens), Chervil (Anthriscus cerefolium), Chicory (Cichorium intybus), Chili pepper (Capsicum spp), Chives (Allium schoenoprasum), Cicely, sweet cicely (Myrrhis odorata), Cilantro, coriander greens, coriander herb (Coriandrum sativum), Cinnamon, Indonesian (Cinnamomum burmannii, Cassia vera), Cinnamon, Saigon or Vietnamese (Cinnamomum loureiroi), Cinnamon, true or Ceylon (Cinnamomum verum, C. zeylanicum), Cinnamon, white (Canella winterana), Cinnamon myrtle (Backhousia myrtifolia), Clary, Clary sage (Salvia sclarea), Clove (Syzygium aromaticum), Coriander seed (Coriandrum sativum), Costmary (Tanacetum balsamita), Cuban oregano (Plectranthus amboinicus), Cubeb pepper (Piper cubeba), Cudweed (Gnaphalium spp.), Culantro, culangot, long coriander (Eryngium foetidum), Cumin (Cuminum cyminum), Curry leaf (Murraya koenigii), Curry plant (Helichrysum italicum), Dill seed (Anethum graveolens), Dill herb or weed (Anethum graveolens), Elderflower (Sambucus spp.), Epazote (Dysphania ambrosioides), Fennel (Foeniculum vulgare), Filé powder, gumbo filé (Sassafras albidum), Fingerroot, krachai, temu kuntji (Boesenbergia rotunda), Galangal, greater (Alpinia galanga), Galangal, lesser (Alpinia officinarum), Galingale (Cyperus spp.), Garlic chives (Allium tuberosum), Garlic (Allium sativum), Garlic, elephant (Allium ampeloprasum var. ampelaprasum), Ginger (Zingiber officinale), Ginger, torch, bunga siantan (Etlingera elatior) (Indonesia), Golpar, Persian hogweed (Heracleum persicum) (Iran), Grains of paradise (Aframomum melegueta), Grains of Selim, Kani pepper (Xylopia aethiopica), Horseradish (Armoracia rusticana), Houttuynia cordata (Vietnam), Huacatay, Mexican mangold, mini marigold (Tagetes minuta), Hyssop (Hyssopus officinalis), Indonesian bay leaf, daun salam (Syzygium polyanthum), Jasmine flowers (Jasminum spp.), Jimbu (Allium hypsistum) (Nepal), Juniper berry (Juniperus communis), Kaffir lime leaves, Makrud lime leaves (Citrus hystrix) (Southeast Asia), Kala zeera (or kala jira), black cumin (Bunium persicum) (South Asia), Kawakawa seeds (Macropiper excelsum) (New Zealand), Kencur galangal, kenijur (Koempferia galanga), Keluak, kluwak, kepayang (Pangium edule), Kinh gioi, Vietnamese balm (Elsholtzia ciliate), Kokam seed (Garcinia indica) (Indian confectionery), Korarima, Ethiopian cardamom, false cardamom (Aframomum corrorima) (Eritrea), Koseret leaves (Lippia adoensis) (Ethiopia), Lavender (Lavandula spp.), Lemon balm (Melissa officinalis), Lemongrass (Cymbopogon citratus, C.flexuosus, and other Cymbopogon spp.), Lemon ironbark (Eucalyyptus staigeriana) Australia), Lemon myrtle (Backhousia citriodora) (Australia), Lemon verbena (Lippia citriodora), Leptotes bicolor (Paraguay and southern Brazil), Lesser calamint (Calamintha nepeta), nipitella, nepitella (Italy), Licorice, liquorice (Glycyrrhiza glabru), Lime flower, linden flower (Tilia spp.), Lovage (Levisticum officinale), Mace (Myristica fragrans), Mahlab, St. Lucie cherry (Prunus mahaleb), Marjoram (Origanum majorana), Marsh mallow (Althaea officinalis), Mastic (Pistacia lentiscus), Mint (Mentha spp.) 25 species, hundreds of varieties, Mountain horopito (Pseudowintera colorata) ‘Pepper-plant’ (New Zealand), Musk mallow, abelmosk (Abelmoschus moschatus), Mustard, black, mustard plant, mustard seed (Brassica nigra), Mustard, brown, mustard plant, mustard seed (Brassica juncea), Mustard, white, mustard plant, mustard seed (Sinapis alba), Nasturtium (Tropaeolum majus), Nigella, kalonji, black caraway, black onion seed, Njangsa, djansang (Ricinodendron heudelotii) (West Africa), Nutmeg (Myristica fragrans), Neem, Olida (Eucalyptus olida) (Australia), Oregano (Origanum vulgare, O. heracleoticum, and other species), Orris root (Iris germanica, I.florentina, I. pallida), Pandan flower, kewra (Pandanus odoratissimus), Pandan leaf, screwpine (Pandanus amaryllifolius), Paprika (Capsicum annuum), Paracress (Spilanthes acmella, Soleracea) (Brazil), Parsley (Petroselinum crispum), Pepper: black, while, and green (Piper nigrum), Pepper, Dorrigo (Tasmannia stipitata) (Australia), Pepper, long (Piper longum), Pepper, mountain, Comish pepper leaf (Tasmannia lanceolata), Peppermint (Mentha piperata), Peppermint gum leaf (Eucalyptus dives), Perilla, shiso (Perilla spp.), Peruvian pepper (Schinus molle), Pandanus amaryllifolius, Brazilian pepper or Pink pepper (Schinus terebinthifolius), Quassia (Quassia amara) (bitter spice in aperitifs and some beers and fortified wines), Ramsons, wood garlic (Allium ursinum), Rice paddy herb (Limnophila aromatica) (Vietnam), Rosemary (Rosmarinus officinalis), Rue (Ruta graveolens), Safflower (Carthamus tinctorius), for yellow color, Saffron (Crocus sativus), Sage (Salvia officinalis), Saigon cinnamon (Cinnamomum loureiroi), Salad bumet (Sanguisorba minor), Salep (Orchis mascula), Sassafras (Sassafras albidum), Savory, summer (Satureja hortensis), Savory, winter (Satureja montana), Silphium, silphion, laser, laserpicium, lasarpicium (Ancient Roman cuisine, Ancient Greek cuisine), Shiso (Perilla frutescens), Sorrel (Rumex acetosa), Sorrel, sheep (Rumex acetosella), Spearmint (Mentha spicata), Spikenard (Nardostachys grandiflora or N. jatamansi), Star anise (Illicium verum), Sumac (Rhus coriaria), Sweet woodruff (Galium odoratum), Szechuan pepper, Sichuan pepper (Zanthoxylum piperitum), Tarragon (Artemisia dracunculus), Thyme (Thymus vulgaris), Thyme, lemon (Thymus x citriodorus), Turmeric (Curcuma longa), Vanilla (Vanilla planifolia), Vietnamese cinnamon (Cinnamomum loureiroi), Vietnamese coriander (Persicaria odorata), Voatsiperifery (Piper borbonense), Wasabi (Wasabiajaponica), Water-pepper, smartweed (Polygonum hydropiper), Watercress (Rorippa nasturtium-aquatica), Wattleseed (from about 120 spp. Of Australian Acacia), White mustard (Sinapis alba), Wild betel (Piper sarmentosum) (Southeast Asia), Wild thyme (Thymus serpyllum), Willow herb (Epilobium parviflorum), Winter savory (Satureja montana), Wintergreen (Gaultheria procumbens), Wood avem, herb bennet (Geum urbanum), Woodruff (Galium odoraium), Wormwood, absinthe (Artemisia absinthium), Yellow mustard (Brassica hirta=Sinapis alba), Yerba buena, any of four different species, many unrelated, Za'aiar (herbs from the genera Origanum, Calamintha, Thymus, and/or Satureja), Zedoary (Curcuma zedoarta), and the like, and mixtures thereof.

As used herein, the term “pharmaceutically acceptable” refers to counter-ions, compounds, materials, ingredients, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio. Therefore, the composition may refer to a combination of an active ingredient, the nutraceutical composition, and/or a carrier, mert or active, making the composition especially suitable for diagnostic or therapeutic use in vivo or ex vivo.

In certain embodiments, the nutraceutical composition of the present disclosure in any of its embodiments may further comprise (v) a pharmaceutically acceptable carrier or excipient. As used herein, the phrase “pharmaceutically acceptable carrier or excipient” refers to a pharmaceutically acceptable material, composition, or vehicle such as a liquid or solid filler, diluent, binder, manufacturing aid (e.g. lubricant, talc magnesium, calcium or zinc stearate, or steric acid), or solvent encapsulating material, involved in carrying or transporting the subject composition from one organ, or portion of the body, to another organ, or portion of the body. Each carrier must be “acceptable” in the sense of being compatible with the other ingredients of the formulation and not injurious to a patient.

As used herein, a “pharmaceutically acceptable carrier” refers to a carrier or diluent that does not cause significant irritation to an organism, does not abrogate the biological activity and properties of the administered active ingredient or composition, and/or does not interact in a deleterious manner with the other components of the composition in which it is contained. As used herein, the term “carrier” encompasses any excipient, binder, diluent, filler, salt, buffer, stabilizer, solubilizer, lipid, or other material well known to one of ordinary skill in the art for use in pharmaceutical formulations. The choice of a carrier for use in a composition will depend upon the intended route of administration for the composition. The preparation of pharmaceutically acceptable carriers and formulation containing these materials is well known to those of ordinary skill in the art. Examples of physiologically acceptable carriers include buffers such as phosphate buffers, citrate buffer, and buffers with other organic acids; antioxidants including ascorbic acid; low molecular weight (preferably less than about 10 residues) polypeptides; proteins, such as serum albumin, gelatin or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter-ions such as sodium, and/or nonionic surfactants such as TWEEN® (ICI, Inc.; Bridgewater, N.J.), polyethylene glycol (PEG), and PLURONICS™ (BASF; Florham Park, N.J.). Additional exemplary materials which can serve as pharmaceutically acceptable carriers include, but are not limited to; (1) sugars, such as lactose, glucose and sucrose; (2) starches, such as corn starch and potato starch; (3) cellulose, and its derivatives, such as sodium carboxy methyl cellulose, ethyl cellulose and cellulose acetate; (4) powdered tragancanth; (5) malt; (6) gelatin; (7) talc; (8) excipients, such as cocoa butter and suppository waxes; (9) oils, such as peanut oil, cottonseed oil, safftower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) other non-toxic compatible substances employed in pharmaceutical formulations and mixtures thereof.

As used herein, an “excipient refers to an inert substance added to a composition to further facilitate administration of a compound. Examples of excipienls include, but are not limited to, calcium carbonate, calcium phosphate, various sugars and types of starch, cellulose derivatives, gelatin, vegetable oils, and polyethylene glycols.

In another embodiment, wetting agents emulsifiers and lubricants, such as sodium lauryl sulfate and magnesium stearate, as well as coloring agents, release agents, coating agents, sweetening, flavoring and perfuming agents, preservatives and antioxidants may also be present in the compositions described herein. Exemplary pharmaceutically acceptable antioxidants include, but are not limited to: (1) water soluble antioxidants, such as ascorbic acid, cysteine hydrochloride, sodium bisulfate, sodium metabisulfite, sodium sulfite, and the like; (2) oil soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetracetic acid (EDTA), sorbitol, tartaric acid, phosphoric acid, and the like.

In another embodiment, the pharmaceutically acceptable carrier or excipient is a binder. As used herein “binder” refers to materials that hold the ingredients in a tablet formulation together. Binders ensure that tablets and granules can be formed with the required mechanical strength, and give volume to low active dose tablets. Exemplary pharmaceutically acceptable binders include, but are not limited to: (1) saccharides and their derivatives, such as sucrose, lactose, starches, cellulose or modified cellulose such as microcrystalline cellulose, carboxymethyl cellulose, and cellulose ethers such as hydroxypropyl cellulose (HPC), and sugar alcohols such as xylitol, sorbitol, or maltilol; (2) proteins such as gelatin; and (3) synthetic polymers including polyvinylpyrrolidone (PVP) and polyethylene glycol (PEG).

Binders can additionally be classified according to their application. Solution binders are dissolved in a solvent (i.e. water or alcohol in wet granulation processes). Exemplary solution binders include, but are not limited to, gelatin, cellulose, cellulose derivatives, polyvinylpyrrolidone, starch, sucrose, and polyethylene glycol. Dry binders are added to the powder blend, either after a wet granulation step, or as part of a direct powder compression (DC) formula. Exemplary dry binders include, but are not limited to, cellulose, methyl cellulose, polyvinylpyrrolidone and polyethylene glycol. In terms of the present disclosure, the pharmaceutically acceptable carrier or excipient may be a solution binder, a dry binder or mixtures thereof.

In one embodiment, the nutraceutical composition is formulated for systemic administration. Formulations of the present disclosure include, but are not limited to, those suitable for oral, nasal, topical (i.e. buccal and sublingual), rectal and/or parenteral administration. The formulations may conveniently be presented in unit dosage form and may be prepared by any methods well known in the art of pharmacy. As used herein, the “active ingredient” of the nutraceutical composition formulation refers to the nutraceutical composition as described herein in any of its embodiments. The amount of active ingredient, nutraceutical composition as described herein in any of its embodiments, which can be combined with a carrier material to produce a single dosage form, will vary depending upon the host being treated as well as the particular mode of administration. The amount of active ingredient, the nutraceutical composition as described herein in any of its embodiments, which can be combined with a carrier material to produce a single dosage form, will generally be that amount of the nutraceutical composition as described herein which produces a therapeutic effect and which does not cause systemic toxicity, for example through sepsis or a cytokine cascade.

According to a second aspect, the present disclosure relates to a method of increasing at least one reproductive indicator selected from the group consisting of sperm concentration, spenn count, and progressive sperm motility, the method comprising administering to a male human subject in thereof an effective dosage of a nutraceutical composition to increase at least one reproductive indicator selected from the group consisting of sperm concentration, sperm count and progressive sperm motility, the nutraceutical composition comprising (i) garden cress, (ii) nigella sativa, (iii) commiphora myrrha, and (iv) fenugreek, wherein each constituent (i), (ii), (iii), and (iv) is present in an effective amount and an effective proportion relative to the total amount of (i)-(iv) such that when administered to the male human subject the nutraceutical composition is effective in increasing at least one reproductive indicator selected from the group consisting of sperm concentration, sperm count, and progressive sperm motility relative to the reproductive indicator prior to the administering. In a preferred embodiment, the method comprises a nutraceutical composition as described herein in any of its embodiments.

In a preferred embodiment, the method of the present disclosure in any of its embodiments increases at least one indicator in the male human subject selected from the group consisting of sperm concentration, spenn count, and progressive sperm motility by 40-80% relative to the at least one reproductive indicator in the male human subject selected from the group consisting of sperm concentration, sperm count, and progressive sperm motility prior to the administering, preferably by 45-78%, preferably by 50-76%, preferably by 55-74%, preferably by 60-72%, preferably 65-70% relative to the at least one reproductive indicator in the male human subject selected from the group consisting of sperm concentration, sperm count, and progressive sperm motility prior to the administering. In certain embodiments, sperm count and/or sperm concentration in the male human subject is increased by at least 25% after the administering, preferably at least 30%, preferably at least 40%, preferably at least 50%, preferably at least 60%, preferably at least 70%, preferably at least 80% after the administering. In certain embodiments, progressive sperm motility in the male human subject is increased by greater than 5 percentage points after the administering, preferably greater than 10 percentage points, preferably greater than 15 percentage points, preferably greater than 20 percentage points, preferably greater than 25 percentage points, preferably greater than 30 percentage points, preferably greater than 40 percentage points after the administering.

In a preferred embodiment, sperm count in the male human subject is greater than 40,000,000 per ejaculate after the administering, preferably greater than 45,000,000, preferably greater than 50,000,000, preferably greater than 55,000,000, preferably greater than 60,000,000, preferably greater than 65,000,000, preferably greater than 70,000,000, preferably greater than 80,000,000, preferably greater than 90,000,000, preferably greater than 100,000,00 per ejaculate after the administering. In a preferred embodiment, sperm concentration in the male human subject is greater than 10,000.000 per mL of ejaculate after the administering, preferably greater than 12,000,000, preferably greater than 14,000,000, preferably greater than 16,000,000, preferably greater than 18,000,000, preferably greater than 20,0000,000, preferably greater than 25,000,000 per mL of ejaculate after the administering.

In a preferred embodiment, progressive sperm motility in the male human subject is greater than 25% after the administering, preferably greater than 30%, preferably greater than 35%, preferably greater than 40%, preferably greater than 45%, preferably greater than 50%, preferably greater than 60% after the administering. In a preferred embodiment, non-progressive sperm motility and/or sperm immotility is reduced by at least 5 percentage points after the administering, preferably at least 10 percentage points, preferably at least 15 percentage points, preferably at least 20 percentage points, preferably at least 25 percentage points after the administering. In a preferred embodiment, sperm demonstrating abnormal forms of morphology (i.e. abnormal heads, midpieces, and/or principle pieces) are reduced by at least 5 percentage points after the administering, preferably at least 10 percentage points, preferably at least 15 percentage points, preferably at least 20 percentage points, preferably at least 25 percentage points, preferably at least 30 percentage points, preferably at least 35 percentage points, preferably at least 40 percentage points, preferably at least 45 percentage points, preferably at least 50 percentage points after the administering.

The dosage and treatment duration are dependent on factors, such as bioavailability of an active ingredient, administration mode, toxicity of an active ingredient, gender, age, lifestyle, body weight, the use of other drugs and dietary supplements, the disease or condition stage, tolerance and resistance of the body to the administered active ingredient, and the like and may then be determined and adjusted accordingly. In certain embodiments, the nutraceutical composition in any of its embodiments is administered in a dose in a range of 1-1000 mg/kg based on the weight of the subject, preferably 2-500 mg/kg, preferably 5-250 mg/kg, preferably 10-100 mg/kg, preferably 15-80 mg/kg, preferably 20-50 mg/kg based on the weight of the subject. In certain embodiments, the nutraceutical composition in any of its embodiments is administered at various dosages (i.e. a first does at 50 mg/kg and a second dose at 10 mg/kg). In certain embodiments, the interval of time between the administration of the nutraceutical composition and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months, 5 months, 6 months, 1 months, 8 months, 9 months, 10 months, 11 months, 12 months, 1 year, 2 years, or any period of time in between. In certain embodiments, the nutraceutical composition and one or more additional therapies are administered less than 1 day, 1 week, 2 weeks, 3 weeks, 4 weeks, 1 month, 2 months, 3 months, 6 months, 1 year, 2 years, or 5 years apart.

In a preferred embodiment, the nutraceutical composition of the present disclosure in any of its embodiments is administer at a dosage in the range of 0.9-10 mg at least once per day, preferably 1-9.5 mg, preferably 2-9 mg, preferably 5-8.5 mg, preferably 6-8 mg, preferably 6.5-7.5 mg, or about 7.0 mg at least once per day, for example 4.5-5 mg. In a preferred embodiment, the administering is performed 1-5 times daily, preferably 2-4 times daily, or about 3 times daily. In a preferred embodiment, the administering is performed 1-5 times daily, preferably 2-4 times daily, or about 3 times daily for a time period of 5-150 days, preferably 10-100 days, preferably 15-50 days, preferably 18-45 days, preferably 20-40 days, preferably 25-30 days, preferably 30-40 days.

As used herein, the terms “administer”, “administering”, “administration” and the like refer to the methods that may be used to enable delivery of an active ingredient and/or the nutraceutical composition as described herein in any of its embodiments to a desired site of biological action. Routes or modes of administration are as set forth herein. These exemplary methods include, but are not limited to, oral routes, intraduodenal routes, parenteral injection (including intravenous, subcutaneous, intraperitoneal, intramuscular, intravascular, or infusion), topical and rectal administration. Administration techniques that cau be employed with the nutraceutical compositions and methods described herein will be familiar to those of ordinary skill in the art and are not viewed as particularly limiting.

In a preferred embodiment, the administering is performed in an enteral manner, most preferably the administering is performed orally. As used herein, enteral administration of enteral nutrition refers to feeding or active ingredient administration by the digestion process of a gastrointestinal (GI) tract, such as for example the human gastrointestinal tract. Enteral administration involves the esophagus, stomach, and small and large intestines. Methods of administration include, oral, sublingual (dissolving under the tongue), and rectal. The route of administration is important as it affects drug metabolism, drug clearance, and thus dosage. Enteral administration may be divided into three different categories, depending on the entrance point into the GI tract oral (by mouth), gastric (through the stomach), and rectal (from the rectum). Gastric introduction typically involves the use of a tube through the nasal passage (NG tube) or a tube in the belly leading directly to the stomach (PEG) tube. Exemplary methods of oral administration are solids including, but not limited to, pills, tablets, time release technology, and osmotic controlled release capsules and liquids including, but not limited to, solutions, softgels, suspensions, emulsions, syrups, elixirs, tinctures, and hydrogels. Exemplary methods of rectal administration include, but are not limited to, ointments, suppositories, enemas, murphy drips, and nutrient enemas. The mechanism of adsorption from the intestine is typically passive transfer or carrier-mediated transport. Compositions administered in an enteral manner may be subjected to significant first pass metabolism, and therefore, the amount of active ingredient entering the systemic circulation following administration may vary significantly for different individuals and active ingredients.

In certain embodiments, the nutraceutuical composition in any of its embodiments has various release states (i.e. controlled release or immediate release). As used herein, immediate release refers to the release of an active ingredient or composition substantially immediately upon administration. In one embodiment, immediate release occurs when there is dissolution of an active ingredient or composition within 1-20 minutes after administration. Dissolution can be of all (full) or less than all (partial) of the active ingredient or composition, such as, for example about 70%, about 75%, about 80%, about 85%, about 90%, about 91%, about 92%, about 935. about 94%, about 95%, about 96%, about 97%, about 98%, about 99%, about 99.5%, about 99.9%. or about 99.99% of the active ingredient or composition. In one embodiment, immediate release results in complete or less than complete dissolution within about 1 hour following administration. Dissolution can be in a subject's stomach and/or intestine. In one embodiment, immediate release results in dissolution of an active ingredient within 1-20 minutes after entering a subject's stomach. For example, dissolution of 100% of an active ingredient can occur in the prescribed time. In one embodiment, immediate release results in complete or less than 1 hour following rectal administration. In some embodiments, immediate release is through inhalation, such that dissolution occurs in a subject's lungs.

As used herein, controlled release or sustained release, refers to the release of an active ingredient from a composition of dosage form in which the active ingredient or composition is released over an extended period of time. In one embodiment, controlled release results in dissolution of an active ingredient within 20-180 minutes after entering the stomach. In one embodiment, controlled release occurs when there is dissolution of an active ingredient within 20-180 minutes after being swallowed. In one embodiment, controlled release occurs when there is dissolution 20-180 minutes after entering the intestine. In one embodiment, controlled release results in substantially completer dissolution after at least 1 hour following administration. In one embodiment, controlled release results in substantially complete dissolution after at least 1 hour following oral administration. In one embodiment, controlled release results in substantially complete dissolution after at least 1 hour following rectal administration. In one embodiment, the composition is not a controlled release composition.

Depending upon the intended mode of administration (oral, parenteral, topical, etc.), the composition may be in the form of solid, semi-solid, or liquid dosage forms, including, but not limited to tablets, suppositories, pills, capsules, powders, liquids or suspensions, preferably in unit dosage form suitable for a single administration of a precise dosage.

Solid dosage forms for oral administration may include, bus are not limited to capsules, tablets, pills, powders, and granules. In such solid dosage forms, the active ingredient and/or nutraceutical composition is typically combined with one or more adjuvants appropriate to the indicated route of administration. If administered by way of mouth (per os), the active ingredient and/or nutraceutical composition may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone and/or polyvinyl alcohol, and then tableted or encapsulated for convenient administration. Such capsules or tablets may contain a controlled release formulation as can be provided in a dispersion of active ingredient or composition in hydroxypropylmethyl cellulose. In certain embodiments, the capsule and/or tablet does not contain a controlled release formulation. In the case of capsules, tablets, and pills, the dosage forms may also comprise buffering ingredients including, but not limited to, sodium citrate, magnesium or calcium carbonate or bicarbonate. Additionally, tablets, capsules, and/or pills may additionally be prepared with enteric coatings. In a preferred embodiment, the nutraceutical composition of the present disclosure in any of its embodiments is administered in the form of an encapsulated powder.

Liquid dosage forms for oral administration can include pharmaceutically acceptable emulsions, solutions, suspensions, syrups, and elixirs containing inert diluent commonly used in the art, such as, for example, water. Such compositions may also comprise adjuvants including, but not limited to, wetting ingredients, emulsifying and suspending ingredients, and sweetening, flavoring, and/or perfuming ingredients.

For therapeutic purposes, formulations for parenteral administration may be in the form of aqueous or non-aqueous isotonic sterile injection solutions or suspensions. As used herein, “parenteral” includes, but is not limited to, intravenous, intravesical, intraperitoneal, subcutaneous, intramuscular, intralesional, intracranial intrapulmonal, intracardial, intrasternal, and sublingual injections, or infusion techniques. These solutions and/or suspensions may be prepared from sterile powders or granules optionally having one or more of the carriers or diluents mentioned for use in the formulations for oral administration. The active ingredient and/or composition may be dissolved in water, polyethylene glycol, propylene glycol, ethanol, corn oil, cottonseed oil, peanut oil, sesame oil, benzyl alcohol, sodium chloride, and/or various buffers. Further envisaged adjuvants and modes of administration are well and widely known to those in the pharmaceutical art.

Injectable preparations, such as, for example, sterile injectable aqueous or oleaginous suspensions can be formulated according to the known art using suitable dispersing or wetting ingredients, and/or suspending ingredients. In other embodiments, the sterile injectable preparation may also be a sterile injectable solution or suspension in a non-toxic parenterally acceptable diluent or solvent, such as, for example, as a solution in 1,3-butanediol. Exemplary, acceptable vehicles and solvents that can be employed include, but are not limited to, water, Ringer's solution, and an isotonic sodium chloride solution. In addition, sterile, fixed oil may be conventionally employed as a solvent or suspending medium. For this purpose any bland fixed oil may be employed such as, for example, synthetic mono- or diglycerides. Additionally, fatty acids, such as, for example oleic acid, may find use in the preparation of injectables. Dimethyl acetamide, surfactants, including ionic and non-ionic detergents, and polyethylene glycols may also be employed. Mixtures of solvents and wetting ingredients such as those discussed above are also employed.

Suppositories for rectal administration may be prepared by mixing the active ingredient and or composition with a suitable non-irritating excipient, such as, for example, cocoa butter, synthetic mono-, di- or triglycerides, fatty acids, and polyethylene glycols that are ideally solid at room temperatures but liquid at the rectal temperature and thus will melt in the rectum allowing release of the drug. Further, topical administration may also involve the use of transdermal administration, such as, for example transdermal patches and/or iontophoresis devices. Various acceptable forms of drug formulations will be well known to those of ordinary skill in the art.

In certain embodiments, the administration is stopped once a subject is treated.

According to a third aspect, the present disclosure relates to a nutraceutical beverage for treating male infertility, the beverage comprising (i) garden cress, (ii) nigella sativa, (iii) commiphora myrrha, (iv) fenugreek, and (vi) a liquid, wherein each constituent (i), (ii), (iii), and (iv) is present in an effective amount and an effective proportion relative to the total amount of (i)-(iv) such that when administered to a male human subject in need thereof the nutraceutical beverage is effective in increasing at least one reproductive indicator selected from the group consisting of sperm concentration, sperm count, and progressive sperm motility relative to the reproductive indicator prior to the administering. In a preferred embodiment, the nutraceutical beverage comprises a nutraceutical composition as described herein in any of its embodiments.

As used herein, the term beverage means a consumer reads product comprising any liquid (vi) intended for human or animal consumption. Exemplary types of nutraceutical beverages include, but are not limited to, berries and small fruit juices, smoothies, citrus fruit juices, dairy products (i.e. milks, yogurts, soy beverages), grape juices, syrups, wines, beers, teas, coffees, cocoas/chocolates, tomato juices, and the like. In preferred embodiments, the liquid (vi) is an aqueous liquid. As used herein, an aqueous liquid refers to any liquid having significant water content. The aqueous liquid may contain additional ingredients without deviating from the meaning of aqueous liquid as used herein. In certain embodiments, the components (i)-(iv) may be partially or fully dissolved in the liquid (v). The aqueous liquid may include any other ingredients so as to produce a desired benefit, flavor or effect. Exemplary other ingredients may include, but are not limited to, vitamins, natural and artificial flavors, electrolytes, dietary fiber, calcium, iron, zinc, calcium disodium EDTA, citric acid, CO₂ or any other desired ingredient.

In a preferred embodiment, the nutraceutical beverage of the present disclosure in any of its embodiments has a weight ratio of total weight of (i)-(iv) in grams to total volume of the nutraceutical beverage in the range of 1:5 to 1:100, preferably 1:10 to 1:80, preferably 1:30 to 1:70, preferably 1:40 to 1:60.

In a preferred embodiment, the nutraceutical beverage is a tea, an aromatic beverage commonly prepared by pouring hot or boiling water over a plant or plant parts. In certain embodiments, the nutraceutical composition and/or nutraceutical beverage of the present disclosure in any of its embodiments may further comprise one or more tea plants, including herbal tea plants, in addition to components (i)-(iv). Exemplary tea plants include, but are not limited to, Anise tea (seeds or leaves), Asiatic penny-wort leaf, Artichoke tea, Bee Balm, Boldo, Burdock, Caraway tea, Catnip tea, Cbamomile tea, Che Dang tea (Ilex causue leaves), Chinese knot-weed tea, Chrysanthemum tea, Cinnamon, Coca tea, Coffee tea leaves and coffee cherry tea, Cerasse, Citrus peel (including bergamot, lemon and orange peel), Dandelion coffee, Dill tea, Echinacea tea, Elderberry, European Mistletoe (Viscum album), Essiac tea, Fennel, Gentian, Ginger root, Ginseng, Goji, Hawthorn, Hibiscus, Ho Yan Hor Herbal Tea, Honeybush, Horehound, Houttuynia, Hydrangea tea (Hydrangea serrata Amacha), Jiaogulan, Kapor tea, Kava root, Kratom, Kuzuyu, Labrador tea, Lapacho (also known as Taheebo). Lemon Balm, Lemon and ginger tea, Lemon grass, Luo han guo, Licorice root, Lime blossom, Mint Mountain Tea, Neem leaf, Nettle leaf, New Jersey Tea, Noni tea, Oksusu cha, Pennyroyal leaf, Pine tea, Qishr, Red clover tea, Red raspberry leaf, Roasted barley tea, Roasted wheat, Rooibos (Red Bush), Rose hip, Roselle petals (species of Hibiscus: aka Bissap, Dah, etc.), Rosemary, Sagebrush, California Sagebrush, Sage, Sakurayu, Salvia, Scorched rice, Skullcap, Sereudib (tea), Sobacha, Spicebush (Lindera benzoin), Spruce tea, Staghorn sumac fruit, Stevia, St. John's Wort, Tea (Camellia sinensis), Thyme, Tulsi, Holy Basil, Uncaria tomentosa, commonly known as Cat's Claw, Valerian, Verbena (Vervains), Vetiver, Wax gourd, Wong Lo Kat, Woodruff, Yarrow, and the like and mixtures thereof.

The examples below are intended to further illustrate protocols for preparing and administering the nutraceutical compositions of the present disclosure. Further, they are intended to illustrate assessing the properties of these compositions and assessing their performance in increasing symptomatic levels of male infertility. They are not intended to limit the scope off the claims.

### EXAMPLE 1

### Preparation and Administration of the Nutraceutcal Composition

In order to prepare the composition, the prime ingredients, garden cress, nigella sativa (black cumin), commiphora myrrha (molmol), and fenugreek were cleaned to remove all physical impurities. The ingredients were then individually ground to obtain a fine powder, optionally using filters. The nutraceutical composition was prepared batchwise by mixing 140 g of garden cress, 140 g of nigella sativa, 20 g of commiphora myrrha, and 90 g of fenugreek. The mixture was ground and mixed together to form a preferably homogeneous mixture that was then encapsulated in an amount of about 1 mg per capsule. The capsules were then administered to patients orally once daily for a period of 21 days. Table 1 presents the semen analysis of a patient before the treatment and after the treatment.

TABLE 1
Comparative semen analysis following treatment with the nutraceutical composition

| | Result | Result | | Reference | |
|---|---|---|---|---|---|
| | (Before Treatment) | (After Treatment) | Unit | Range | |
| Test | | | | | |
| Volume | 5.0 | 5.0 | mL | ≥1.5 | mL |
| Appearance | Normal | Normal | | Normal | |
| Viscosity | Normal | Normal | | Normal | |
| Color | G. White | G. White | | G. White | |
| pH | 8.0 | 8.0 | | ≥7.2 | |
| Liquefaction | 30 | 30 | min. | ≤60 | min. |
| Sperm Count/mL | 8,000,000 | 13,500,000 | | ≥15 | million |
| Total Sperm Count/ejaculate | 40,000,000 | 67,500,000 | | ≥39 | million |
| Motility (1 hour) | 55 | 60 | % | ≥40% | |
| Motility Grade | | | | | |
| Progressive (PR) | 30 | 50 | % | ≥32% | |
| None Progressive (Np) | 25 | 10 | % | | |
| Immotile | 45 | 40 | % | | |
| Normal Forms | 5 | 45 | % | ≥4% | |
| Abnormal beads | 30 | 25 | % | | |
| Abnormal Midpieces | 15 | 15 | % | | |
| Abnormal Principle Pieces | 45 | 10 | % | | |
| Excess Residual Cytoplasm | 5 | 5 | % | | |
| WBCs/mL | 480,000 | 320,000 | | ≤1 | milliion |

| | | | |
|---|---|---|---|
| Agglutination | Absent | Absent | Absent |

Thus, the foregoing discussion disclose and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1: A nutraceutical composition for treating male infertility, the composition comprising:
(i) garden cress;
(ii) nigella sativa;
(iii) commiphora myrrha; and
(iv) fenugreek;
wherein each constituent (i), (ii), (iii) and (iv) is present in an effective amount and an effective proportion relative to the total amount of (i)-(iv) such that when administered to a male human subject in need thereof the nutraceutical composition is effective in increasing at least one reproductive indicator selected from the group consisting of sperm concentration, sperm count, and progressive sperm motility relative to the reproductive indicator prior to the administering.

2: The nutraceutical composition of claim 1, further comprising (v) a pharmaceutically acceptable carrier or excipient.

3: The nutraceutical composition of claim 1, which has a wet basis moisture content of less than 15% relative to the total weight of the nutraceutical composition.

4: The nutraceutical composition of claim 1, which is in a powder form and has an average particle size of 0.1-2.5 mm.

5: The nutraceutical composition of claim 1, wherein a weight ratio of garden cress to nigella sativa to commiphora myrrha to fenugreek is in the range of 5-10:5-10:0.25-2.0:2-6.

6: The nutraceutical composition of claim 1, wherein a weight ratio of garden cress to nigella sativa to commiphora myrrha to fenugreek is 7:7:1:4.5.

7: A method of increasing at least one reproductive indicator selected from the group consisting of sperm concentration, sperm count, and progressive sperm motility, the method comprising:
administering to a male human subject in need thereof an effective dosage of a nutraceutical composition to increase at least one reproductive indicator selected from the group consisting of sperm concentration, sperm count, and progressive sperm motility, the nutraceutical composition comprising:
(i) garden cress;
(ii) nigella sativa;
(iii) commiphora myrrha; and
(iv) fenugreek;
wherein each constituent (i), (ii), (iii) and (iv) is present in an effective amount and an effective proportion relative to the total amount of (i)-(iv) stich that when administered to the male human subject the nutraceutical composition is effective in increasing at least one reproductive indicator selected from the group consisting of sperm concentration, sperm count, and progressive sperm motility relative to the reproductive indicator prior to the administering.

8: The method of claim 7, wherein the nutraceutical composition further comprises (v) a pharmaceutically acceptable carrier or excipient.

9: The method of claim 7, wherein the nutraceutical composition has a weight ratio of garden cress to nigella sativa to commiphora myrrha to fenugreek in the range of 5-10:5-10:0.25-2.0:2-6.

10: The method of claim 7, wherein the nutraceutical composition has a weight ratio of garden cress to nigella sativa to commiphora myrrha to fenugreek of 7:7:1:4.5.

11: The method of claim 7, wherein the administering is performed in an enteral manner.

12: The method of claim 7, wherein the nutraceutical composition is administered in the form of an encapsulated powder.

13: The method of claim 7, wherein the nutraceutical composition is administered at a dosage in the range of 1-10 mg at least once per day.

14: The method of claim 7, wherein the administering is performed 1-5 times daily.

15: The method of claim 7, wherein the administering is performed 1-5 times daily for a time period of 5-150 days.

16: The method of claim 7, which increases at least one reproductive indicator in the male human subject selected from the group consisting of sperm concentration, sperm count, and progressive sperm motility by 40-80% relative to the at least one reproductive indicator in the male human subject selected from the group consisting of sperm concentration, sperm count, and progressive sperm motility prior to the administering.

17: A nutraceutical beverage for treating male infertility, the beverage comprising:
(i) garden cress;
(ii) nigella sativa:
(iii) commiphora myrrha;
(iv) fenugreek; and
(vi) a liquid;
wherein each constituent (i), (ii), (iii) and (iv) is present in an effective amount and an effective proportion relative to the total amount of (i)-(iv) such that when administered to a male human subject in need thereof the nutraceutical beverage is effective in increasing at least one reproductive indicator selected from the group consisting of sperm concentration, sperm count, and progressive sperm motility relative to the reproductive indicator prior to the administering.

18: The nutraceutical beverage of claim 17, wherein a weight ratio of garden cress to nigella sativa to commiphora myrrha to fenugreek is in the range of 5-10.0:5-10:0.25-2.0-2.6.

19: The nutraceutical beverage of claim 17, wherein a weight ratio of garden cress to nigella sativa to commiphora myrrha to fenugreek is 7:7:1:4.5.

20: The nutraceutical beverage of claim 17, wherein a ratio of total weight of (i)-(iv) in grams to total volume of the nutraceutical beverage is in the range of 1:5 to 1:100.
